(19) United States
(12) Patent Application Publication
(10) Pub. No.: US 2009/0176306 A1
(43) Pub. Date: Jul. 9, 2009

(54) METHOD OF INDUCING DIFFERENTIATION FROM VISCERAL PREADIPOCYTE TO VISCERAL ADIPOCYTE

(76) Inventors: Toshio Taira, Hokkaido (JP); Kyoko Shimizu, Hokkaido (JP); Masato Sakai, Hokkaido (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/916,701

(22) PCT Filed: Jun. 12, 2006
(86) PCT No.: PCT/JP2006/312197

§ 371 Date: Feb. 20, 2009

(30) Foreign Application Priority Data
Jun. 10, 2005 (JP) 2005-171219

(51) Int. Cl. C12N 5/06

(52) U.S. Cl. 435/377; 435/325; 435/408

(57) ABSTRACT

The invention provides a method of inducing differentiation of a visceral preadipocyte and a method of culturing a visceral adipocyte comprising culturing the cell in a medium containing serum free of heparin-adsorbable components. The invention also provides for the removal of heparin-adsorbable components by, for example, applying serum to a heparin affinity column thereby adsorbing the heparin-adsorbable component to heparin. A visceral adipocyte cultured by the method of the present invention is useful in a study of differentiation, growth and metabolism of an adipocyte, elucidation of the mechanism of development and progress of diseases such as diabetes, hyperlipemia, hypertension and arteriosclerosis, and development of a drug for preventing and treating such a disease.

### RELATED APPLICATIONS

This application claims priority of JP Appln No. 2005-171219, filed Jun. 10, 2005, the contents of which are herein incorporated by reference.

### FIELD OF THE INVENTION

The present invention relates to a method of culturing a visceral adipocyte, a method of inducing differentiation of a visceral preadipocyte and a medium used in such methods.

### BACKGROUND ART

There are four major types of mammalian adipocytes: a brown adipocyte that produces heat in a biological system; a white adipocyte that stores energy; a bone marrow adipocyte present in the bone marrow, whose role has not been well elucidated yet; and a visceral adipocyte present in the abdominal cavity. Among these adipocytes, a white adipocyte present under the skin has been widely studied in terms of a method of inducing differentiation to an adipocyte and a substance for inhibiting differentiation (JP-A-2000-157260 and JP-A-2002-138045, all hereby incorporated by reference in its entirety).

The visceral adipocyte has been suggested to be associated with so-called lifestyle-related diseases, such as diabetes, hyperlipemia, hypertension and arteriosclerosis (Anat Rec Part A 272A: 398-402, 2003, and J. Vet. Med. Sci. 63 (1): 17-23, 2001, all hereby incorporated by reference in its entirety). However, studies on the differentiation and metabolism of the visceral adipocyte have been largely lacking.

### SUMMARY OF THE INVENTION

The present invention provides a method of inducing differentiation of a visceral preadipocyte to a visceral adipocyte comprising obtaining a visceral preadipocyte and culturing the visceral preadipocyte in a medium containing serum free of heparin-adsorbable components. The serum may be any conventional serum such as calf serum, including neonatal calf serum or fetal calf serum.

The present invention provides a serum free of heparin-absorbable components by contacting the serum with a heparin-immobilized support, thereby allowing the heparin-adsorbable component to be adsorbed to heparin. In an embodiment, the heparin-immobilized support is a heparin affinity column.

The present invention also provides a medium for inducing differentiation from a visceral preadipocyte to a visceral adipocyte and a medium for culturing a visceral adipocyte wherein the medium comprises serum free of heparin-adsorbable components.

In addition, the present invention provides a method of preparing a medium for inducing differentiation from a visceral preadipocyte to a visceral adipocyte and a medium for culturing a visceral adipocyte. This method comprises obtaining a heparin-immobilized support, contacting serum with the heparin-immobilized support, collecting a serum free of heparin-adsorbable components, and mixing the serum with a basal medium for cell culture.

The present invention also provides a method of culturing a visceral adipocyte. In another aspect, the present invention provides a visceral adipocyte cultured by the method of the present invention.

The medium provided by the present invention permits the differentiation of visceral preadipocytes. In addition, a visceral adipocyte differentiated and/or cultured by the method of the present invention permits the development of a drug that impacts the differentiation, growth and/or metabolism of an adipocyte, thereby providing for the development of drug to prevent or treat diseases associated with visceral adipoctyes, such as diabetes, hyperlipemia, hypertension and arteriosclerosis.

### BRIEF DESCRIPTION OF THE DRAWINGS

FIG. 1 shows a chromatogram of NCS fractionated on a heparin affinity column.

FIG. 2 shows a chromatogram of the heparin-adsorbed fraction of NCS, which is fractionated using a salt concentration gradient on a heparin affinity column.

### DETAILED DESCRIPTION OF THE INVENTION

Despite the interest in visceral adipocytes and the association of such cells with life-style related diseases such as diabetes, hyperlipemia, hypertension, and arteriosclerosis, visceral adipocytes have not been the subject of much study. The inventors believe that such studies have not been conducted at least in part because of the lack of a primary cell culture system in which a visceral adipocyte is stably maintained as well as the lack of a differentiation induction system.

The present inventors previously found that differentiation of a visceral adipocyte can be efficiently induced by isolating a visceral preadipocyte from rat mesentery and culturing it in a medium containing micellized oil and fat. However, it was observed that the degree of differentiation or growth rate varied depending on the conditions for isolating a visceral adipocyte or the lot of the serum to be added to the culture medium. Accordingly, the inventors sought a stable method of inducing differentiation and of growing such cells.

The present inventors found that one or more of the components in serum inhibit differentiation from a visceral preadipocyte to a visceral adipocyte, and that differentiation to a visceral adipocyte can be induced by using a medium containing serum from which this component or components have been removed. Accordingly, the present inventors provide a method of inducing differentiation of a visceral preadipocyte to a visceral adipocyte by obtaining a visceral preadipocyte and culturing the visceral preadipocyte in a medium comprising a serum free of heparin-adsorbable components. The medium comprising a serum free of heparin-adsorbable components may also be used to culture such visceral adipocytes.

The term “serum free of heparin-adsorbable components” as used herein means serum obtained by a process of removing heparin-absorbable components. Preferably 50%, more preferably 60%, 70%, 80% or 90%, most preferably 100% of the heparin-adsorbable components have been removed. The term “heparin-adsorbable components” as used herein means one or more substances in serum that can bind to heparin when serum and heparin are brought into contact with each other in a buffer (e.g. 0.02M phosphate buffer, pH7.4) containing 0-0.15M NaCl. As known in the art, heparin is a general term that refers to a mucopolysaccharide whose skeletal structure comprises a repeating structure of a disaccharide unit of D-glucosamine and L-iduronic acid residues linked at the 1,4-positions. Heparin has a heterogeneous structure with a number of sulfate groups. Heparin is widely used as a blood coagulation inhibitor. It is also used in protein separation or a study of protein interaction analysis based on its binding property to various proteins.

As shown in the Examples below, the inventors have found that the heparin-adsorbable components comprise one or more substances that may inhibit differentiation of a visceral preadipocyte. Accordingly, the present inventors provide methods for differentiating visceral preadipocytes and for culturing the differentiated visceral adipocyte in a medium that is free of heparin-adsorbable components.

The serum to be used in the present invention may be any type of serum that is commercially available for cell culture. Among them, calf serum (CS), fetal calf serum (FCS) or newborn calf serum (NCS) can be used, with an embodiment directed to NCS. NCS is generally serum obtained from a calf at one week to one year after birth, though it also varies depending on the supplier.

To remove the heparin-adsorbable component from serum, serum is brought into contact with heparin in any means known in the art. One embodiment uses a water-insoluble support on which heparin is immobilized. Any support commonly used in the art may be used as the support for immobilizing heparin, for example, polystyrene, polyacrylamide, and cellulose or a derivative thereof. In one embodiment, the invention uses a heparin affinity column prepared by filling a column with a heparin-immobilized support. A variety of heparin affinity columns of various capacities are commercially available for protein separation or analysis, and any of these columns may also be used in the method of the present invention.

In one embodiment, the serum is introduced to the immobilized heparin. Separation of the serum from the immobilized heparin removes the heparin-adsorbable component and provides a serum free of heparin-adsorbable components remains. In one embodiment, the heparin-adsorbable component is adsorbed to heparin at conventional salt concentration of about 0.15 M NaCl and is eluted at a salt concentration of about 0.5 M NaCl.

In such an embodiment, for example, the heparin column is previously equilibrated with a conventional buffer (e.g., PBS) with a salt concentration equal to that of serum (generally about 0.15 M NaCl). The serum is applied to the column, then a buffer with the same salt concentration is applied and a flow-through fraction is collected that constitutes serum free of the heparin-adsorbable component. Thereafter, for example, to study the absorbed components, the heparin-adsorbable component may be eluted from the column by applying a buffer with a salt concentration of about 0.5 M NaCl.

Once obtained, the serum free of heparin-absorbable components may be concentrated by, for example, ultrafiltration, and mixed with any commonly used basal medium. Such basal medium includes, for example, αMEM, F-12, SF02, PRMI 1640 and opti-MEM medium. The serum treated with heparin can be added to a basal medium at a concentration of from about 1% to about 30%, preferably 5%, 10%, 15% or 20%.

In another embodiment, oil and fat is added to the medium of the present invention. Any of a plant oil and fat such as soybean oil or palm oil, an animal oil and fat such as lard or beef tallow, and a mineral oil such as paraffin may be used. In an embodiment, oil and fat derived from a plant is used, such as those containing abundance of palmitic acid, stearic acid, oleic acid, linoleic acid and linolenic acid. The oil and fat may be added to the medium in the form of an emulsion. The emulsified oil and fat can be prepared by vigorously mixing or sonicating the oil and fat with a commonly used emulsifier such as egg-yolk lecithin in an aqueous solution.

In yet another embodiment, the medium of the present invention contains no or little, if any, indomethacin, dexamethasone, or a PPAR-γ agonist that is known as an agent for stimulating differentiation of an adipocyte. When such a substance is present in the medium, it might disturb the process of differentiation of an adipocyte and the effect of a differentiation inhibitor or a differentiation inducer.

A visceral preadipocyte can be obtained from any tissue such as mesenteric adipose tissue, omental adipose tissue, epididymis peripheral adipose tissue, kidney peripheral adipose tissue, or uterus peripheral adipose tissue. For example, mesenteric adipose tissue in the abdominal cavity is excised from a rat, washed, minced with scissors and dispersed in a buffer, and then treated with collagenase or other proteolytic enzyme such as dispase or trypsin. The obtained mixture is centrifuged to remove floating cells, and the precipitate contains visceral preadipocytes. The visceral preadipocytes are seeded at about 0.1-5.0×10⁵ cells/ml into the medium of the present invention containing serum free of heparin-adsorbable components. When culturing the visceral preadipocytes in a cell culture container containing the medium of the invention, they will be induced to differentiate to a visceral adipocyte within 4-10 days.

Any containers commonly used in cell culture may be used for culturing the visceral preadipocytes, for example, a dish, a flask, and a roller bottle. Cultivation can be carried out under standard conditions commonly used for culturing mammalian cells, for example, at 37° C. under 5% CO₂. The medium is replaced everyday or once per several days. In this way, a primary culture of visceral adipocytes can be obtained and maintained in the differentiated state.

Differentiation to a visceral adipocyte may be conventionally monitored by observing a cell with the naked eye or under a microscope to examine the presence of, for example, a fat globule in a cell. It may also be monitored by determining the level of triglyceride in cytoplasm, measuring fat synthesis activity (GPDH), or measuring the amount of mRNA of PPAR-γ. Such measurement methods have been established in the art (see, for example, Kozak, L. P., and Jensen, J. T. (1974) J. Biol. Chem. 249, 7775-7781, hereby incorporated by reference in its entirety).

The inventors intend the above description to provide those of ordinary skill in the art with a complete disclosure of how to make and how to use the present invention and do not intend the description to limit the scope of what the inventors regard as their invention. Moreover, while the present invention is described with reference to the specific embodiments, those skilled in the art will understand that various changes may be made and equivalents may be substituted without departing from the true spirit and scope of the invention. In addition, many modifications can be made to adapt to a particular situation, material, composition of matter, process, process step or steps, to the objective, spirit, and scope of the present invention. All such modifications are intended to be within the scope of the claims appended hereto. In addition, while the specification may describe certain advantages of the invention, if those advantages are not included in the claims, they should be considered per se limitations to the claimed invention.

The inventors also note that, when ranges are cited, the invention encompasses each intervening value between the upper and lower limits of the range, unless the context clearly indicates otherwise. Moreover, the invention also encompasses ranges excluding either or both of the upper and lower limits of the range, unless specifically excluded from the stated range. The inventors also note that, in accordance with the usual meaning of “a” and “the” in patents, reference, for example, to “a” cell or “the” cell is inclusive of one or more cells. In addition, unless otherwise indicated, all numbers expressing quantities of ingredients, reaction conditions, and so forth used in the specification and claims are to be understood as being modified in all instances by the term “about.” Thus, unless indicated to the contrary, the numerical parameters set forth in the specification and claims are approximations that may vary depending upon the standard deviation found in their respective testing measurements. At the very least, each numerical parameter as set forth in the claims should at least be construed in light of the number of reported significant digits and by applying ordinary rounding techniques.

The specification is most thoroughly understood in light of the cited references, all of which are hereby incorporated by reference in their entireties. And, unless defined otherwise, the meanings of all technical and scientific terms used herein are those commonly understood by one of ordinary skill in the art to which this invention belongs.

Hereinafter, the present invention will be described in more detail with reference to Examples, however, the present invention is not limited to these Examples. The inclusion of these examples is not intended to represent that the experiments below are all or the only experiments performed. Efforts have been made to ensure accuracy with respect to numbers used (for example, amounts, temperature, etc.) but some experimental errors and deviations should be accounted for.

### Example 1

Mesenteric adipose tissue in the abdominal cavity was excised from male Sprague-Dawley rats at 3 to 5 weeks of age (average body weight: 100 g) and washed three times with Hanks balanced salt solution. The adipose tissue was minced with scissors and incubated at 37° C. for 40 minutes in DMEM/F-12 medium containing 0.2% collagenase and 1.0% bovine albumin. Then, the tissue suspension treated with collagenase was filtered through a mesh with a pore size of 600 μm, and Hanks balanced salt solution was added. This mixture was centrifuged at 800×g for 10 minutes, and precipitate was washed three times with Hanks balanced salt solution and once with DMEM/F-12 medium, and then filtered through a mesh with a pore size of 100 μm.

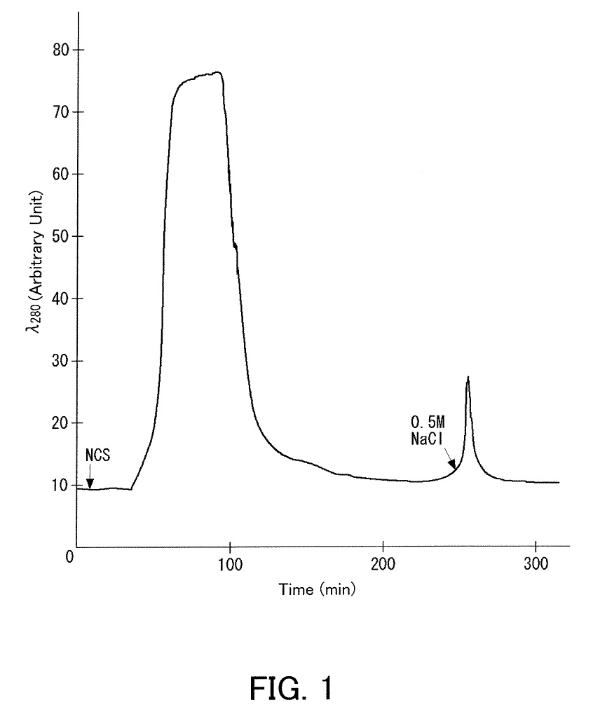

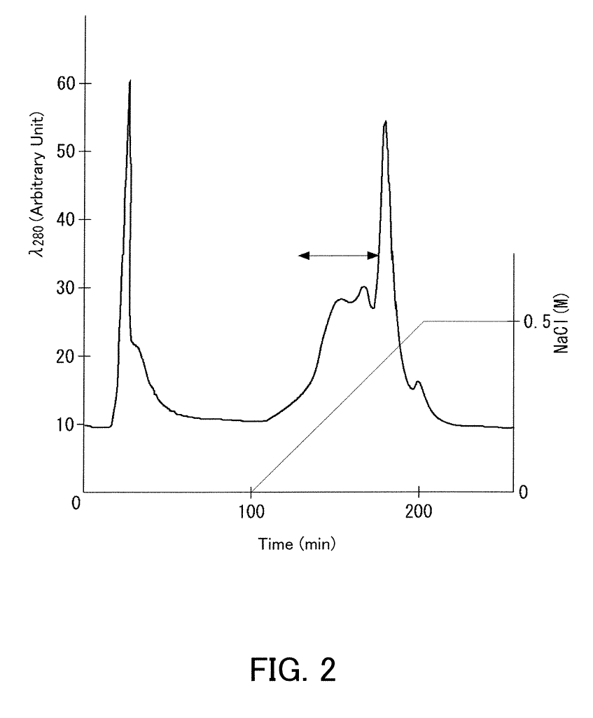

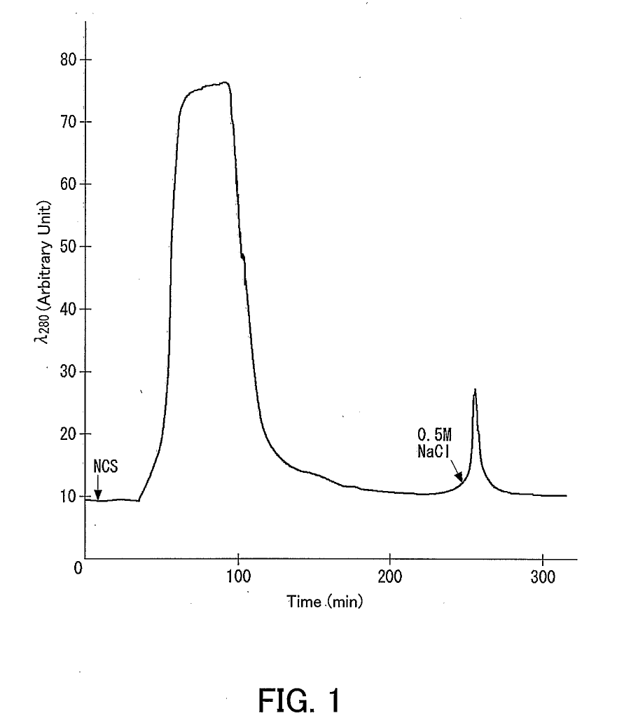

Onto a heparin column (heparin agarose, Pharmacia) with a capacity of 320 ml which had been equilibrated with PBS (0.15 M NaCl), 250 ml of NCS was charged. PBS was applied to the column and a flow-through fraction was collected, which was NCS free of heparin-absorbed components. Subsequently, PBS containing 0.5 M NaCl was applied to the column to elute a heparin-adsorbed fraction (FIG. 1).

150 ml of the NCS free of heparin-absorbed components was concentrated to 100 ml with an ultrafilter with a 10000 MW cutoff, and the concentrate was sterilized through a filter with a pore size of 0.2 μm. A 20 ml portion of the sterilized concentrate was added to 180 ml of DMEM/F-12 medium to prepare the medium. As a control, a medium was prepared by adding 20 ml of NCS which had not been treated with the heparin column to 180 ml of DMEM/F-12 medium.

The cell suspension prepared from mesenteric adipose tissue in the abdominal cavity as described above was seeded on a dish at 0.5×10⁵ cells/cm², and cultured at 37° C. under 5% CO₂ in the medium containing the serum free of heparin-adsorbable components or in the control medium. The medium was replaced everyday or once per two days.

After 4 days in culture, morphological evaluation of differentiation of an adipocyte was carried out by observing the cell under a microscope to examine the presence of a fat vacuole in the cytoplasm. The level of fat generated by the cell was determined by Oil-Red O staining. A predetermined number of cultured cells were fixed with 20% formaldehyde and stained with Oil-Red O. After the cells were washed with PBS, the dye was extracted with isopropanol, and the concentration of the dye was measured by absorbance at 540 nm.

When the medium containing serum free of heparin-adsorbable components was used, differentiation from a visceral preadipocyte to a visceral adipocyte was observed. On the other hand, when the control medium was used, only a few cells differentiated but most of the cells remained as preadipocyte, suggesting that a factor that inhibits differentiation to a visceral adipocyte is contained in the heparin-adsorbed fraction of serum.

### Example 2

A serum free of heparin-adsorbable components was prepared in the same manner as in Example 1, except that fetal calf serum (FCS) was used instead of neonatal calf serum (NCS). The fraction was added to DMEM/F-12 medium and visceral preadipocytes were cultured in the medium. Differentiation from a visceral preadipocyte to a visceral adipocyte was observed in the same manner as in Example 1.

### Example 3

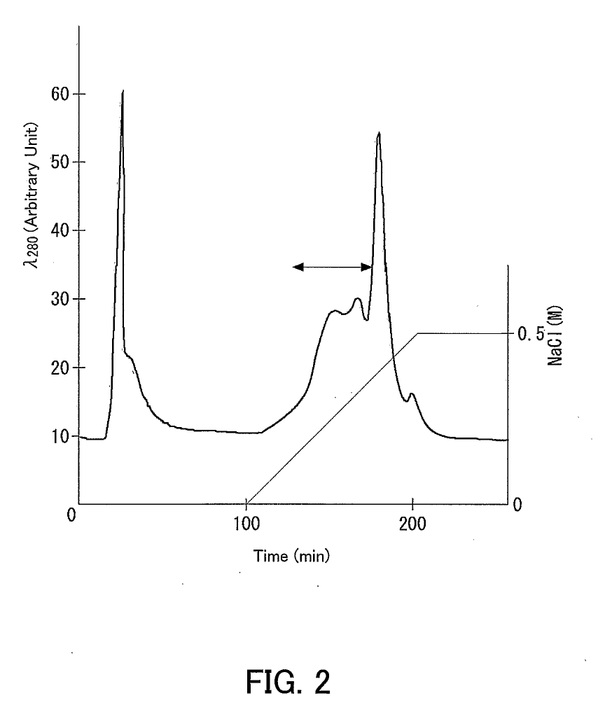

A heparin-adsorbed fraction eluted with 0.5 M NaCl obtained in Example 1 was equilibrated with a buffer containing 0.02 M disodium hydrogen phosphate (pH 7.4). 10 ml of this solution was applied to a heparin column with a capacity of 50 ml which had been equilibrated with 0.02 M phosphate buffer (pH 7.4). The column flow rate was set at 15 ml/min, and detection was carried out by absorbance at 280 nm. After a flow-through fraction was collected, the column was washed with 0.02 M phosphate buffer (pH 7.4), and then eluted with a linear concentration gradient of 0-0.5 M NaCl in 0.02 M phosphate buffer (pH 7.4). The chromatogram is shown in FIG. 2.

A fraction eluted from 130 to 170 minutes (a fraction shown by ←→ in the figure) was collected and desalted. A 20 ml portion of the desalted solution was added to 180 ml of DMEM/F-12 medium, and examined for its effect on differentiation of a visceral adipocyte in the same manner as in Example 1. As a result, it was found that this fraction has a strong inhibitory activity on differentiation of a visceral adipocyte.

From the above results, it was shown that a factor that inhibits differentiation of a visceral adipocyte is contained in the heparin-adsorbable component of serum, and that differentiation of a visceral adipocyte can be induced by culturing a visceral preadipocyte using serum free of heparin-adsorbable components.

### INDUSTRIAL APPLICABILITY

The medium provided by the present invention permits the differentiation of visceral preadipocytes. In addition, a visceral adipocyte differentiated and/or cultured by the method of the present invention permits the development of a drug that impacts the differentiation, growth and/or metabolism of an adipocyte, thereby providing for the development of drug to prevent or treat diseases associated with visceral adipoctyes, such as diabetes, hyperlipemia, hypertension and arteriosclerosis.

What is claimed is:

1. A method of inducing differentiation of a visceral preadipocyte to a visceral adipocyte comprising culturing the visceral preadipocyte in a medium containing serum free of heparin-adsorbable components.

2. A method of culturing a visceral adipocyte comprising culturing the visceral adipocyte in a medium containing serum free of heparin-adsorbable components.

3. The method according to claim 1 or 2, wherein the serum is neonatal calf serum.

4. The method according to claim 1 or 2, wherein the serum is fetal calf serum.

5. The method according to any one of claims 1 to 4, wherein the heparin-adsorbable component is removed by bringing serum into contact with a heparin-immobilized support, thereby adsorbing the heparin-adsorbable component to heparin.

6. The method according to claim 5, wherein the heparin-immobilized support is a heparin affinity column.

7. The method according to claim 5, wherein the heparin-adsorbable component is a substance that is adsorbed to heparin at a salt concentration of about 0.15 M NaCl and is eluted at a salt concentration of about 0.5 M NaCl.

8. A visceral adipocyte obtained by a method according to any one of claims 1 to 7.

9. A medium for inducing differentiation from a visceral preadipocyte to a visceral adipocyte comprising serum free of heparin-adsorbable components.

10. A medium for culturing a visceral adipocyte, comprising serum free of heparin-adsorbable components.

11. The medium according to claim 9 or 10, wherein the serum is neonatal calf serum.

12. The medium according to claim 9 or 10, wherein the serum is fetal calf serum.

13. The medium according to any one of claims 9 to 12, wherein the heparin-adsorbable component has been removed by bringing serum into contact with a heparin-immobilized support thereby adsorbing the heparin-adsorbable component to heparin.

14. The medium according to claim 13, wherein the heparin-immobilized support is a heparin affinity column.

15. The medium according to claim 13, wherein the heparin-adsorbable component is a substance that is adsorbed to heparin at a salt concentration of about 0.15 M NaCl and is eluted at a salt concentration of about 0.5 M NaCl.

16. A method of preparing a medium for inducing differentiation from a visceral preadipocyte to a visceral adipocyte comprising contacting serum with a heparin-immobilized support, collecting serum free of heparin-adsorbable components, and mixing the serum with a basal medium for cell culture.

17. A method of preparing a medium for culturing a visceral adipocyte comprising contacting serum with a heparin-immobilized support, collecting a serum free of heparin-adsorbable components, and mixing the serum with a basal medium for cell culture.

18. The method according to claim 16 or 17, wherein the serum is neonatal calf serum.

19. The method according to claim 16 or 17, wherein the serum is fetal calf serum.
